(19) United States
(12) Patent Application Publication
(10) Pub. No.: US 2005/0261591 A1
(43) Pub. Date: Nov. 24, 2005

(54) Image guided interventions with interstitial or transmission ultrasound

(75) Inventors: Emad Boctor, Baltimore, MD (US); Gabor Fichtinger, Bethesda, MD (US)

Correspondence Address: MCKENNA LONG & ALDRIDGE LLP, 1900 K STREET, NW, WASHINGTON, DC, 20006, US

(73) Assignee: THE JOHNS HOPKINS UNIVERSITY, Baltimore, MD (US)

(21) Appl. No.: 11/124,117

(22) Filed: May 9, 2005

Related U.S. Application Data
(63) Continuation-in-part of application No. 10/895,397, filed on Jul. 21, 2004.
(60) Provisional application No. 60/569,004, filed on May 7, 2004.
(60) Provisional application No. 60/577,788, filed on Jun. 8, 2004.
(60) Provisional application No. 60/488,941, filed on Jul. 21, 2003.

(51) Int. Cl. A61B 8/14

(52) U.S. Cl. 600/462

(57) ABSTRACT

Disclosed is a system and method for providing image guidance for medical procedures in which an interstitial ultrasound probe is inserted into the tissue surrounding a tumor. The interstitial ultrasound probe is designed to “ride” in the surrounding tissue so that it may move in conjunction with the tumor and provide intra-operative imagery. A surgeon may guide a surgical instrument and perform interventions such as ablation while tracking the tumor in the ultrasound imagery. The position and orientation of the interstitial ultrasound probe and the surgical instrument are measured throughout the medical procedure to enable either the surgeon, or a robotic arm, to guide the surgical instrument such that it may move in conjunction with the tumor.

This application claims the benefit of U.S. Provisional Patent Application No. 60/569,004, filed on May 7, 2004; U.S. Provisional Patent Application No. 60/577,788, filed on Jun. 8, 2004; and U.S. Non-provisional application Ser. No. 10/895,397 titled ROBOTIC 5D ULTRASOUND SYSTEM, which claims priority to U.S. Provisional Patent Application No. 60/488,941, filed Jul. 21, 2003, all of which are hereby incorporated by reference for all purposes as if fully set forth herein.

The efforts associated with the subject matter of this patent application were supported by the National Science Foundation under grant no. EEC9731478.

### BACKGROUND OF THE INVENTION

1. Field of the Invention

The present invention involves the field of ultrasound imagery. More particularly, the present invention involves a system and method for the use of ultrasound to provide real-time imaging for guiding surgical tools.

2. Discussion of the Related Art

Computer Integrated Surgery has revolutionized surgical procedures, whereby imagery is used to enable a surgeon to more precisely and accurately position surgical tools within a patient. Computer Integrated Surgery has been used to improve surgical procedures such as radiotherapy and various interventions such as ablation, biopsy, and resectioning. In a typical intervention, the surgeon must identify, target, and treat a tumor within a surrounding tissue. According to the related art, images of the tumor and the surrounding tissue are acquired to provide for planning of the procedure, and to provide periodic feedback to the surgeon regarding the state of the tumor and the surrounding tissue at various times throughout the procedure.

As used herein, the term “tumor” may refer to any type of growth or object of interest disposed in or on surrounding tissue, and may refer to a single tumor or multiple tumors located within the same surrounding tissue. The term “intervention” refers to a process within a surgical procedure in which a lesion is treated, such as ablation, thermal therapy, and radiation seed implantation. The term “surgery” refers to the overall procedure, including preparation, surgical tool insertion, intervention, and surgical tool removal. Further, the term “imagery” may refer to a single image or multiple images acquired by a given imaging modality.

Various medical imaging modalities are used to assist in procedure planning, such as Magnetic Resonance Imaging (MRI) and Computer Tomography (CT). These pre-operative imaging modalities are used to identify the tumor within the surrounding tissue so that the surgeon may decide how to most effectively and safely approach and treat the tumor. The tumor is identified within the surrounding tissue by use of automated segmentation techniques, or by manual operator selection. The identification process may be performed as part of the pre-operative imaging process, or may be done as a separate process by different personnel. Pre-operative imagery may be acquired days or weeks before the procedure. CT and MRI generally require large equipment in dedicated facilities, which may make them unsuitable for use in an operation room setting. Further, the pre-operative imaging modality, such as CT, may subject the patient to harmful radiation. Accordingly, it may not be feasible to acquire and process pre-operative imagery in a real-time setting.

Once the medical procedure has been planned using pre-operative imaging, intra-operative imaging is used to provide imagery of the tumor and surrounding tissue during the procedure. In general, related art approaches to intra-operative imaging use ultrasound due that fact that it is easy to use, provides real-time imagery, and is generally unobtrusive in an operating room setting.

One related art intra-operative approach involves transcutaneous ultrasound (TCUS) whereby the TCUS probe is placed against the patient's skin during the procedure. This approach, although it has the benefit of being non-invasive, generally provides poor image quality, depending on the presence of intervening soft tissue and bone between the TCUS probe and the tumor and its surrounding tissue. Different layers of intervening soft tissue and bone provide acoustic interfaces that may obscure the tumor and attenuate the ultrasound signal, thereby decreasing the image quality.

Another related art approach to intra-operative imagery involves the invasive use of ultrasound whereby the surgeon opens the patient to expose the tissue surrounding the tumor, and periodically places an ultrasound probe, like a TCUS probe, against the surrounding tissue. Since this approach mitigates the signal degradation and obscuration problem cuased by the intervening soft tissue and bone, the image quality is generally excellent. However, this approach is extremely invasive. Further, the ultrasound probe must be constantly removed and repositioned on the surrounding tissue to provide room for the surgical instruments. Accordingly, this approach only provides intermittent intra-operative imagery, whereby the surgeon only acquires imagery between interventions. Another invasive related art approach involves the use of laparoscopic ultrasound, which requires anesthesia and considerable preparation during surgery.

Another problem that happens to be common to both the invasive and non-invasive ultrasound approaches is the movement of surrounding tissue during a procedure. Certain surrounding tissue, such as a liver or kidney, may have sufficient elasticity such that a tumor may move on the order of centimeters during surgery.

Intra-operative imagery currently involves a tradeoff between image quality and the invasiveness of image acquisition. Further, intra-operative imagery generally does not provide for real-time image tracking of the surrounding tissue during interventions.

### SUMMARY OF THE INVENTION

Accordingly, the present invention is directed to image guided interventions with interstitial or transmission ultrasound that substantially obviates one or more of the problems due to limitations and disadvantages of the related art. In general, the present invention achieves this by providing real time imagery of a tumor and its surrounding tissue, in a minimally invasive fashion, such that the imagery tracks the movement of the tumor within the patient to better guide surgical tools during intervention. The imagery tracks the movement of the tumor by having an interstitial ultrasound probe located in near proximity to the tumor such that the probe moves in conjunction with the tissue surrounding the tumor.

An advantage of the present invention is that it is minimally invasive.

Another advantage of the present invention that it may assist in robotic surgery by enabling a robotically-guided surgical tool to track the motion of a tumor.

Another advantage of the present invention is that it enables a surgeon to track a tumor undergoing intervention and assess the effectiveness of the intervention.

Another advantage of the present invention is that it provides real time guidance for inserting surgical instruments that compensates for motion of the tumor and its surrounding tissue.

Another advantage of the present invention is that it provides intra-operative imagery of a tumor with minimal image obscuration or signal attenuation due to intervening tissue or acoustic interfaces.

Additional advantages of the invention will be set forth in the description which follows, and in part will be apparent from the description, or may be learned by practice of the invention. The objectives and other advantages of the invention will be realized and attained by the following written description and claims hereof as well as the appended drawings. The aforementioned and other advantages are achieved by an image-based guidance system for medical procedures. The system comprises a first interstitial ultrasound probe, the first interstitial ultrasound probe having a transducer, wherein the interstitial ultrasound probe is configured to be inserted into a patient such that an object of interest is within a field of view of the transducer; and a data system having a computer readable medium encoded with a program for locating and tracking the object of interest according to the position and orientation of the transducer.

The aforementioned and other advantages are achieved by an image-based guidance system for medical procedures. The system comprises a first interstitial ultrasound probe, the first interstitial ultrasound probe having a transducer, herein the interstitial ultrasound probe is configured to be inserted into a patient such that an object of interest is within a field of view of the transducer; and a data system having a computer readable medium encoded with a program for locating and tracking the object of interest according to the position and orientation of the transducer.

The aforementioned and other advantages are achieved by an image-based guidance system for medical procedures. The system comprises a first interstitial ultrasound probe, the first interstitial ultrasound probe having a transducer, wherein the interstitial ultrasound probe is configured to be inserted into a patient such that an object of interest is within a field of view of the transducer; and a data system having a computer readable medium encoded with a program for locating and tracking the object of interest according to the position and orientation of the transducer.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates an exemplary system for performing image guided surgical intervention according to the present invention;

FIG. 2 illustrates an exemplary interstitial ultrasound probe according to the present invention;

FIG. 3 illustrates an exemplary process for providing image guidance for surgical procedures according to the present invention;

FIG. 4 illustrates an optimal interstitial probe placement point and how multiple tumors may be imaged and targeted according to the present invention;

FIG. 5 illustrates another exemplary system for performing image guided surgical intervention according to the present invention; and

FIG. 6 depicts the amplitude and times of transmission and reception of ultrasound signals according to an embodiment of the present invention.

### DETAILED DESCRIPTION OF THE ILLUSTRATED EMBODIMENTS

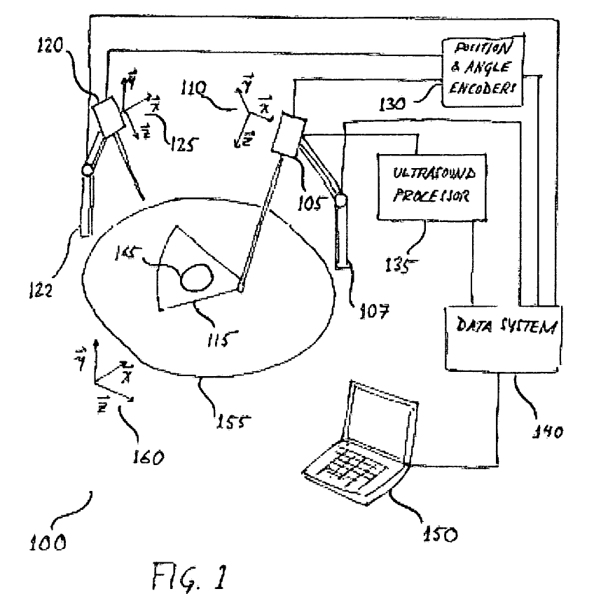

FIG. 1 illustrates an exemplary system 100 according to the present invention. The system 100 includes an interstitial ultrasound probe 105 having a probe reference frame 110 and a field of view 115; a surgical instrument 120 having an instrument reference frame 125; position and angle encoders 130 for measuring the position and orientation of the interstitial ultrasound probe 105 and the surgical instrument 120; an ultrasound processor 135 for providing signals to, and receiving signals from the interstitial ultrasound probe 105; a data system 140; and a user interface 150.

The system 100 may further include a probe mechanical arm 107 for controlling the position and orientation of the interstitial ultrasound probe 105. The probe mechanical arm 107 may be connected to the data system 140 for providing and receiving control signals and data. The position and angle encoders 130 corresponding to the interstitial ultrasound probe 105 may be attached to the probe mechanical arm 107 and may provide measurements corresponding to each degree of freedom of the probe mechanical arm 107.

The system 100 may further include an instrument mechanical arm 122 for controlling the position and orientation of the surgical instrument 120. The instrument mechanical arm 122 may be connected to the data system 140 for providing and receiving control signals and data. The position and angle encoders 130 corresponding to the surgical instrument 120 may be attached to the instrument mechanical arm 122 and may provide measurements corresponding to each degree of freedom of the instrument mechanical arm 122.

The system 100 may be used to perform surgery or interventions on at least one tumor 165 in a surrounding tissue 155 having a tissue reference frame 160. The surrounding tissue, for example, may be a portion of a liver, kidney, or some other anatomical feature.

In this exemplary embodiment, the ultrasound processor 135 includes a ACUSON ultrasound system, and the interstitial ultrasound probe 105 includes an AcuNav™ ultrasound diagnostic catheter, both of which are manufactured by Siemens Medical Solutions, USA, Inc., Ultrasound Division, Issaqua, Wash. However, it will be readily apparent to one of ordinary skill that other ultrasound processors and probes may be used and are within the scope of the invention, provided that the probe may be used interstitially and is capable of being tracked for position and orientation.

The data system 140 may include one or more computers, which may be connected together either locally or over a network. The data system 140 includes software (hereinafter “the software”) for implementing processes according to the present invention. The software may be stored and run on the data system 140, or may be stored and run in a distributed manner between the data system 140, the ultrasound processor 135, and the user interface 150.

In this exemplary embodiment of the present invention, the position and angle encoders 130 include multiple optical markers attached to the interstitial ultrasound probe 105 and the surgical instrument 120, which are tracked using, for example, an OPTOTRAK™ device, manufactured by Northern Digital, Inc. It will be readily apparent to one skilled in the art that alternate devices and systems for providing real-time measurements of position and orientation of the interstitial ultrasound probe 105 and the surgical instrument 120 may be used and are within the scope of the present invention.

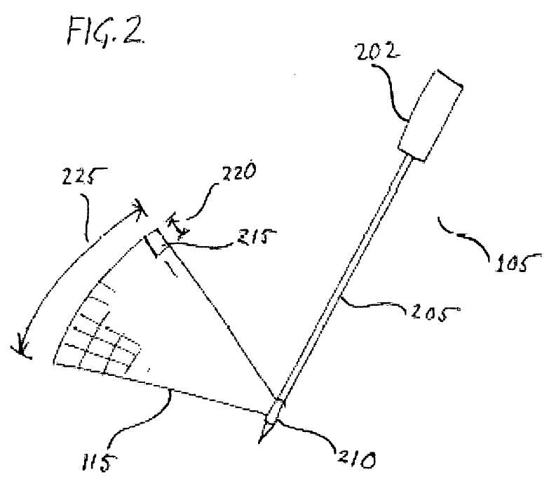

FIG. 2 illustrates an exemplary interstitial ultrasound probe 105 according to the present invention. The interstitial ultrasound probe 105 includes a base 202, a shaft 205 and a transducer 210. The base 202 may include a handle for manual operation or a mounting interface for attachment to a mechanical or robotic arm. The base 202 may also include optical tracking devices that may operate in conjunction with the position and angle encoders 130. Further, the base 202 may include electronics for communicating with the ultrasound processor 135.

The probe reference frame 110 may be defined as fixed relative to the head 202. The shaft 105 is connected to the base 202 and may be substantially rigid to that the location of the transducer 210 may be given in the form of a vector relative to the probe reference frame 110. With the position and orientation of the transducer defined relative to the position and orientation of the probe reference frame 110, the position and orientation of the probe field of view 115 may then be determined by spatially calibrating the interstitial ultrasound probe 105 using ultrasound probe calibration techniques that are known to the art.

The shaft 205 may be rigid such that the position and orientation of the transducer 210 relative to that of the base 202 may be substantially maintained. The diameter of the shaft 205 may be selected based on a tradeoff between maintaining rigidity, minimizing the invasive nature of inserting the interstitial ultrasound probe 105, and providing sufficient interior space within the shaft 205 for wiring between the base 202 and the transducer 210. In a particular embodiment, the outer diameter of the shaft 205 is about 3 mm.

The probe field of view 115 includes the projections of a plurality of pixels 215, which are defined by the characteristics of the transducer 210. For example, the number of elements in the transducer 210 determines the number of pixels 215 along the scan direction 225 of the field of view 115. In a particular embodiment of the interstitial ultrasound probe 105, the transducer has 64 elements. It will be readily apparent to one of ordinary skill that transducers having more elements, and thus more pixels 215 along scan direction 225, are possible and within the scope of the invention. Accordingly, the spatial resolution of the interstitial ultrasound probe 105 corresponds to the number of elements in the transducer 210.

As illustrated in FIG. 2, interstitial ultrasound probe 105 has a side-projected field of view 115. Other field of view 115 orientations are possible, such as an end-projected field of view, and are within the scope of the invention.

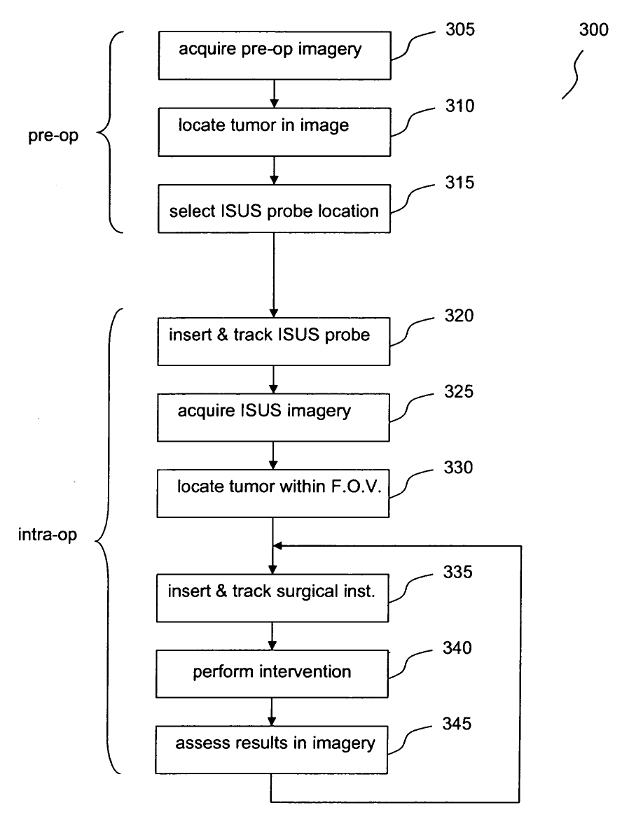

FIG. 3 illustrates an exemplary process 300 for providing ultrasound guided intervention according to the present invention. The process 300 may be divided into a pre-operative phase and an intra-operative phase.

The pre-operative phase includes steps 305-315. The location in which the pre-operative phase is performed depends on the imaging modality to be used for acquiring pre-operative imagery. For instance, CT and MRI are generally performed in dedicated radiology facilities. Also, depending on the circumstances surrounding the procedure to be performed, the pre-operative phase may be optional.

In step 305, pre-operative imagery is acquired, using any combination of imaging modalities, such as MRI or CT. In step 310, the tumor 165 is identified and located in the imagery. Locating may be performed manually, wherein a physician may select the tumor using a cursor and a mouse; or locating it may be performed automatically using image segmentation algorithms, which are known to the art. The result of step 310 is at least one image of the tumor 165 in its surrounding tissue 155. The image may also contain additional anatomical features that may serve as references for the surgeon who will later use, during the surgical procedure, the image to insert the interstitial ultrasound probe 105.

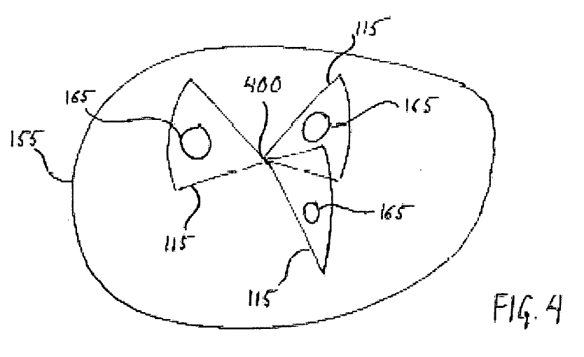

In step 315, with the tumor 165 identified in the image, an optimal target point for the interstitial ultrasound probe 105 is selected. FIG. 4 illustrates an exemplary set of three tumors 165 within a surrounding tissue 155, and a selected target point 400. Target point 400 may be selected such that as many tumors 165 as possible may be placed within the field of view 115 of the interstitial ultrasound probe 105 when the interstitial ultrasound probe 105 is rotated. If there are more tumors 165 than can be seen within field of view 115 of one target point 400, multiple target points 400 may be selected such that they are optimally distributed to image as many tumors 165 as possible while minimizing the number of target points 400. Each additional target point 400 increases the degree of invasiveness of the procedure.

In a particular embodiment, the target point 400 is selected so that the transducer 210 interstitial ultrasound probe 105 will be located within about 7 cm of the tumor, with an optimal distance of about 4 cm. The optimal distance of about 4 cm corresponds to the diverging spatial resolution of the pixels 215 as a function of distance from the transducer 210. At a distance of less than about 4 cm, the field of view 115 may not be sufficiently wide to capture the contours of the tumor 165. At a distance approaching about 7 cm, the signal acoustic signal detected by the transducer 210 becomes attenuated according to the increasing distance, and the ability to discern the contours of the tumor 165 deteriorates since the spatial resolution of the pixels 215 diminishes. It will be readily apparent to one of ordinary skill that these distances are dependent on the characteristics of the transducer 210, and that a transducer 210 with different characteristics, such as number of elements and signal strength, may have a different maximum and optimal distance.

In a particular embodiment, the pre-operative imagery, which shows the located tumor 165 and the selected target point 400, provides the location of the tumor 165 and the target point 400 with an accuracy of about 5 mm. This information will assist the surgeon in determining where to initially insert the interstitial ultrasound probe 105 and the surgical instrument 120.

The intra-operative phase includes steps 320345. The intra-operative phase may be performed in an operating room setting. If the pre-operative phase was performed, the operating room personnel may have pre-operative imagery, which includes the located tumor 165 and the selected target point 400. The pre-operative imagery may be stored in memory on the data system 140.

In step 320, the interstitial ultrasound probe 105 is inserted into the patient in such a manner that the transducer 210 will be placed at the target point 400. The surgeon may have other information to assist in initially guiding the insertion of the interstitial ultrasound probe 105. For example, if pre-operative imagery is available, images of surrounding anatomy in the pre-operative imagery may serve as landmarks for guiding the interstitial ultrasound probe 105. Also, Transcutaneous ultrasound (TCUS) may be used to provide initial guidance for the insertion of the interstitial ultrasound probe 105. A TCUS system is generally available in most operating room settings. Further, TCUS imagery may be registered to the pre-operative imagery by selecting “landmark” features that are visible to both the TCUS and pre-operative imagery. In this case, the volume encompassed by the pre-operative imagery may be registered to the volume imaged by the TCUS in the operating room, and the location of the tumor in the pre-operative imagery may be estimated in the TCUS imagery. Registered TCUS imagery with spatial precision of 5-7 mm may be sufficient to provide initial guidance of the interstitial ultrasound probe 105.

The interstitial ultrasound probe 105 may be inserted robotically through use of the probe mechanical arm 107. In this case, the motion of the probe mechanical arm 107 may be controlled directly by the surgeon, or it may be controlled robotically using motion control software running on the data system 140, which are known to the art

Ultrasound imagery may be acquired by the interstitial ultrasound probe 105 as it is being inserted in step 320. In doing so, the tumor 165 may be identified in the imagery as the interstitial ultrasound probe 105 approaches the target point 400. In this manner, the target point 400 may be refined as the interstitial ultrasound probe 105 is being inserted and located in an optimal position to acquire imagery of the tumors 165. If the tumor 165 is not readily identifiable, the interstitial ultrasound probe 105 may be inserted to the selected target point 400 based on the pre-operative imagery or with other forms of assistance mentioned above.

The interstitial ultrasound probe 105 may be tracked by the position and angle encoders 130 as it is inserted, and after it has reached the target point 400. As mentioned above, given the rigidity of the shaft 205, the position of the transducer 210 may be derived from the position and orientation of the base 202. Also, depending on the type of position and angle encoders 130 used, the shaft 205 may be flexible. For example, if the position and angle encoders 130 include, for example, electromagnetic sensors that are placed close to the rigid part of the transducer 210, then the position and orientation of the transducer may be tracked directly, substantially enabling the shaft 205 to be flexible.

In step 325, ultrasound imagery is acquired by the interstitial ultrasound probe 105. Ultrasound imagery may be acquired throughout the intra-operative phase of process 300. In step 325, the surgeon may rotate the interstitial ultrasound probe 105 to scan the field of view 115 around the axis defined by the shaft 205, thereby generating a 3-dimensional image of the volume surrounding the transducer 210.

In step 330, the tumor 165 is located within the volume defined by the scanned field of view 115. Depending on the nature of the tumor 165 and the surrounding tissue 155, the contours of the tumor may or may not be readily apparent from the imagery. If the tumor 165 is not readily apparent, the surgeon may apply pressure on the interstitial ultrasound probe 105 in order to exert strain on the surrounding tissue 155. In doing so, depending on the differences between the elasticity constant (i.e., Young's modulus) of the tumor 165 and that of the surrounding tissue 155, the tumor 165 may be revealed in the ultrasound imagery by the differences in how it responds to the exerted strain. In this manner, the contours of the tumor 165 may be extracted from the background of the surrounding tissue 155 in the ultrasound imagery.

With the interstitial ultrasound probe 105 located in close proximity to the tumor 165, and given a nearly isotropic Young's modulus for the surrounding tissue 155, there may be an opportunity to perform elastographic analysis on the tumor 165. In general, elastographic analysis requires inducing a 1% strain on the tumor 165. Accordingly, if the tumor 165 is located 5 cm from the transducer 210, a displacement of 0.5 mm, imparted by applying pressure from the interstitial ultrasound probe 105, may be sufficient to perform elastographic analysis.

Once identified in the ultrasound imagery, the contours of the tumor 165 may be extracted from the imagery manually, in which the contours are selected interactively with a cursor and mouse click; or the contours may be ascertained algorithmically using image segmentation techniques that are known to the art. With the contours of the tumor 165 identified, the tumor 165 may be tracked throughout the intra-operative phase of process 300 with successive ultrasound imagery acquired by the interstitial ultrasound probe 105.

With the tumor 165 located, and its contours identified, the surgeon may insert the surgical instrument 120 in step 355. The position and orientation of the surgical instrument 120 may be measured by the position and angle encoders 130 as it is being inserted as well as throughout the intra-operative phase of process 300. Since the position of the transducer 210 is known based on the measured position and orientation of the base 202, the position of the tumor 165 is known relative to the transducer based on the ultrasound imagery, and the position and orientation of the surgical instrument 120 is also known, the surgical instrument 120 may be effectively guided toward the tumor 165. As the surgical instrument 120 approaches the tumor 165, it may enter the field of view 115 of the interstitial ultrasound probe 105, at which point the surgical instrument 120 may be visually guided to the tumor 165 by use of the ultrasound imagery.

The surgical instrument 120 may be inserted manually by a surgeon, or it may be inserted by use of the instrument mechanical arm 122. In this case, the instrument mechanical arm 122 may be directly controlled by the surgeon, or it be controlled robotically using motion control software running on the data system 140. The use of the instrument mechanical arm 122 may enable the coordination of the instrument reference frame 125 with the probe reference frame 110. As the transducer 210 “rides” the surrounding tissue 155 along with the tumor 165, the position and angle encoders 130 measures the motion of the interstitial ultrasound probe 105 by measuring the motion of the base 202, which is rigidly connected to the transducer 210 via the shaft 205. The software running on the data system 140 may acquire the measured motion of the transducer 210 from the position and orientation encoders 130 and use this information to command the instrument mechanical arm 122 to move accordingly. In this case, the surgical instrument 120 may be controlled to track the motion of the tumor 165 while it is being inserted, as well as throughout the intra-operative phase of process 300.

In step 340, once the surgical instrument 120 is properly positioned relative to the tumor, the surgeon may initiate the intervention according to the procedure being performed. In a particular embodiment, the intervention involves ablation, and the surgical instrument 120 is an ablative needle. However, it will be readily apparent to one of ordinary skill that many different procedures, such as radiotherapy and interventions such as biopsy and resectioning, may be performed, and many different surgical instruments 120 may be used, all of which are within the scope of the invention.

In step 345, the surgeon monitors ultrasound imagery acquired by the interstitial ultrasound probe 105 to assess the results of the intervention performed in step 340. Since the tumor 165 has been tracked throughout the intra-operative phase of process 300, changes to the tumor 165 may be apparent. With this information, the surgeon may reposition the surgical instrument, repeat the intervention, and reassess the results of the intervention, thereby iterating steps 335-345. Further, the surgeon may reposition the interstitial ultrasound probe 105 to provide imagery of the tumor from another angle to assist in further interventions of the tumor 165. If there are multiple tumors 165, steps 330-345 may be repeated. Further, if there are multiple target points 400, steps 320345 may be repeated for each target point 400.

Depending on the elasticity of the surrounding tissue 155, the tumor 165 may move throughout the intra-operative phase of process 300. If this happens, the interstitial ultrasound probe 105 may “ride” the surrounding tissue 155 along with the tumor 165. In this case, the position and angle encoders 130 may track the motion of the transducer 210 (and thus track the tumor 165) and provide updated position and angle information for guiding the insertion of the surgical instrument 120.

In an alternate embodiment, a modified system 100, which does not have the probe mechanical arm 107 and the position and angle encoders 130, may be used in a process similar to exemplary process 300. In this embodiment, the interstitial ultrasound probe 105 may be inserted manually. With no position and angle encoders, the tumor 165 may be tracked relatively on an image-by-image basis, as opposed to being tracked absolutely according to an external reference frame. The software may perform image tracking of the tumor 165 by correlating the speckle pattern seen in each image. Speckle refers to observed “texture” in ultrasound imagery, which results from the constructive and destructive interference of acoustic waveforms as they scatter off of small-scale features in tissue. Speckle is stable in ultrasound imagery and may be used to track image motion by use of image correlation algorithms that are known to the art. In performing speckle correlation, relative accuracies of about 5% may be achieved in tracking features such as a tumor 165. This embodiment may be used for the purposes of exploration and diagnostics.

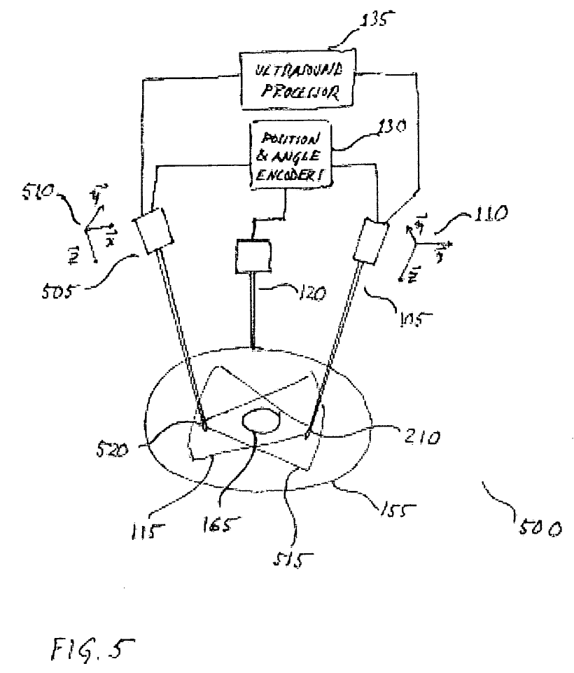

FIG. 5 illustrates another exemplary system 500 according to the present invention. System 500 may have substantially similar components as exemplary system 100, with the addition of a second interstitial ultrasound probe 505 having a second probe reference frame 510 and a transducer 520. The second interstitial ultrasound probe 505 may project a field of view 515.

By incorporating a second interstitial ultrasound probe 505, system 500 may enable the interstitial ultrasound probes to operate in transmission mode, whereby one interstitial ultrasound probe emits acoustic energy, and the other receives the emitted acoustic energy. By synchronizing the signals corresponding to the two interstitial ultrasound probes, the times between transmission and reception, and thus the acoustic propagation between the interstitial ultrasound probes, may be reconstructed. The synchronization between the interstitial ultrasound probes 105 and 505 may be done by software running on the ultrasound processor 135, on the data system 140, or distributed between the two. It will be apparent to one skilled in the art how to control and process the signals from the two interstitial ultrasound probes 105 and 505 such that one may serve as a transmitter and the other as a receiver.

By controlling the interstitial ultrasound probes 105 and 505 such that they are appropriately sequenced, both probes may be used is pulse/echo mode, which is the conventional mode of operation whereby the probe transmits acoustic energy and detects the echo of the same transmitted energy.

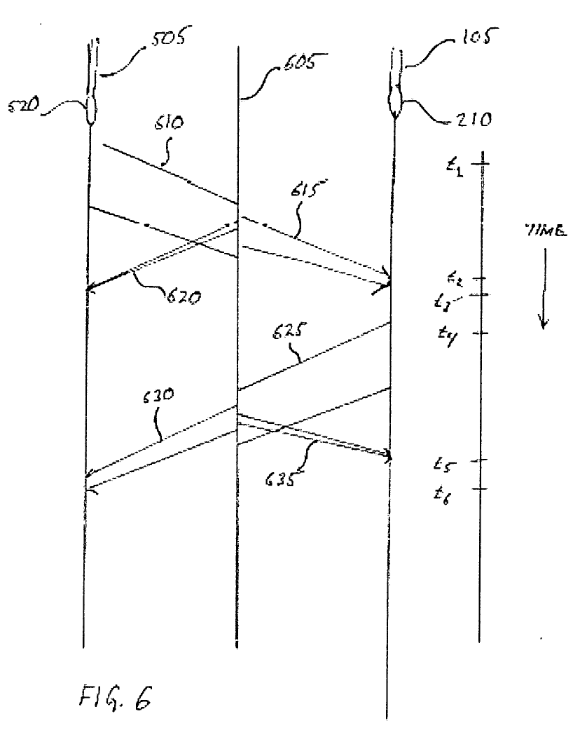

FIG. 6 illustrates an exemplary scenario in which both interstitial ultrasound probes 105 and 505 operate in pulse/echo mode. Each probe receives the echo of its own transmitted acoustic energy as well as the acoustic energy transmitted by the other probe. In FIG. 6, time is depicted in the vertical direction, and spatial distance is depicted in the horizontal direction between interstitial ultrasound probes 505 and 105. An acoustic interface 605 is interposed between interstitial ultrasound probes 105 and 505. The acoustic interface 605 may be a speckle scatterer, a tissue boundary, etc. Solid arrows 610-635 depict acoustic signals propagating through an acoustic medium, whereby the thickness of the arrow corresponds to the amplitude of the acoustic signal. The tapering of arrows 610-635 corresponds to the attenuation of the acoustic signal as it propagates through the acoustic medium.

Referring to FIG. 6, at time t1, transducer 520 transmits an acoustic signal 610 of relatively high amplitude, which is attenuated by the time is hits acoustic interface 605. Some portion of the acoustic signal is transmitted as a signal depicted by arrow 615, which is further attenuated when it is detected by transducer 210 at time t2. A portion of the signal depicted by arrow 615 is scattered by acoustic interface 605, and may scatter in a near isotropic fashion such that only a small portion of the scattered acoustic energy will impinge on transducer 520. The portion of the scattered acoustic energy that is detected by transducer 520 is depicted as signal arrow 620. This signal is further attenuated it is detected by transducer 520 at time t3

At time t4, transducer 210 transmits a signal depicted by arrow 625, and a process substantially similar to that described above is repeated, but in the other direction. By using two interstitial ultrasound probes 105 and 505 in this fashion, each probe receives to signals pertaining to a given acoustic interface 605. The transmitted signal received by the other probe, as depicted by arrows 615 and 630, may have considerably higher amplitude, given the direct propagation of the signal from one transducer to the other. The increased received signal amplitude may enable the two interstitial ultrasound probes 105 and 505 to be placed further apart, which may make it possible to image a larger volume of surrounding tissue 155 and provide imagery of multiple tumors 165 that may be spaced further apart.

In an alternate embodiment, the two interstitial ultrasound probes 105 and 505 may be different such that interstitial ultrasound probe 105 may be a dedicated transmitter and interstitial ultrasound probe 505 may be a dedicated receiver. This may enable each probe to be made smaller, since the transducers 210 may only need to have components for transmitting and transducer 520 may only need to have components for receiving.

In an additional embodiment, the second ultrasound probe may be a transcutaneous ultrasound probe, which may be placed in acoustic contact with the patient. This transcutaneous ultrasound probe may serve as the receiver or the transmitter. However, if the transcutaneous ultrasound probe is used as the receiver, spatial resolution may be improved in that more elements may be used, since the transcutaneous ultrasound probe does not have the size and volume constraint that applies to the interstitial ultrasound probe 105.

Also, elastography may be enabled with the transmission mode ultrasound embodiments because of the increased signal amplitude that is possible with transmission mode. The increased signal amplitude results in improved dynamic range, which may improve the precision of the elastography. By operating in transmission mode, microscopic elasticity features of the tumor may be determined.

It will be readily apparent to one skilled in the art that any of the transmission mode embodiments may be used without the use of mechanical arms or position and angle encoders 130, and may be used for relative image-based tracking as described above.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. An image-based guidance system for medical procedures, comprising:
a first interstitial ultrasound probe, the first interstitial ultrasound probe having a transducer, wherein the interstitial ultrasound probe is configured to be inserted into a patient such that an object of interest is within a field of view of the transducer; and
a data system having a computer readable medium encoded with a program for locating and tracking the object of interest according to the position and orientation of the transducer.

2. The system of claim 1, wherein the first interstitial ultrasound probe includes a substantially rigid shaft.

3. The system of claim 1, further comprising a means for measuring an orientation and position of the first interstitial ultrasound probe.

4. The system of claim 3, wherein the means for measuring the orientation and position of the first interstitial ultrasound probe comprises a position and angle encoder.

5. The system of claim 4, further comprising a first mechanical arm connected to the base of the first interstitial ultrasound probe.

6. The system of claim 5, wherein the computer readable medium is encoded with a program for controlling the position and orientation of the first mechanical arm.

7. The system of claim 3, further comprising:
a surgical instrument; and
a means for measuring an orientation and position of the surgical instrument.

8. The system of claim 7, further comprising a second mechanical arm, the second mechanical arm connected to the surgical instrument.

9. The system of claim 8, wherein the computer readable medium is encoded with a program for controlling a position and orientation of the second mechanical arm based on the orientation and position information corresponding to the first interstitial ultrasound probe.

10. The system of claim 3, further comprising:
a second interstitial ultrasound probe; and
a means for measuring a position and orientation of the second interstitial ultrasound probe.

11. An image-based guidance method for medical procedures, the method comprising:
inserting a first interstitial ultrasound probe into surrounding tissue, the surrounding tissue being proximately located relative to an object of interest;
identifying the object of interest in imagery acquired from the first interstitial ultrasound probe;
measuring a position and orientation of the first interstitial ultrasound probe;
locating and tracking the object of interest based on the position and orientation of the first interstitial ultrasound probe; and
performing a medical procedure on the object of interest using the location and tracking information.

12. The method of claim 11, wherein performing the medical procedure comprises:
inserting a surgical tool into the surrounding tissue, wherein inserting includes compensating for the position and orientation of the first interstitial ultrasound probe; and
performing intervention.

13. The method of claim 12, further comprising assessing the results of the medical procedure, the assessing including monitoring ultrasound imagery acquired from the first interstitial ultrasound probe.

14. The method of claim 11, further comprising selecting a target point within surrounding tissue before inserting a first interstitial ultrasound probe.

15. The method of claim 14, wherein selecting a target point within a surrounding tissue includes selecting a point within a field of view of the first interstitial ultrasound probe.

16. The method of claim 15, wherein selecting a target point within a field of view includes selecting a target point within about 7 cm of the object of interest.

17. The method of claim 11, further comprising acquiring pre-operative imagery of the object of interest and the surrounding tissue.

18. The method of claim 17, wherein acquiring pre-operative imagery includes acquiring MRI imagery.

19. The method of claim 12, wherein inserting a first interstitial ultrasound probe into the surrounding tissue includes controlling a mechanical arm connected to the first interstitial ultrasound probe.

20. The method of claim 11, wherein identifying the object of interest in imagery acquired from the first interstitial ultrasound probe includes:
exerting strain on the surrounding tissue; and
observing a motion of the object of interest in relation to a motion of the surrounding tissue.

21. The method of claim 12, further comprising inserting a second interstitial ultrasound probe into the surrounding tissue.

22. The method of claim 21, wherein identifying the object of interest in imagery acquired from the first interstitial ultrasound probe includes:
synchronizing a signal corresponding to the first interstitial ultrasound probe with a signal corresponding to the second interstitial ultrasound probe;
operating the first and second interstitial ultrasound probes in pulse/echo mode; and
identifying the tumor in a first image acquired by the first interstitial ultrasound probe and a second image acquired by the second interstitial ultrasound probe.

23. The method of claim 17, wherein identifying the object of interest in imagery acquired from the first interstitial ultrasound probe includes:
transmitting acoustic energy from the second interstitial ultrasound probe;
receiving a portion of the acoustic energy by the first interstitial ultrasound probe; and
synchronizing the a signal corresponding to the first interstitial ultrasound probe with a signal corresponding to the second interstitial ultrasound probe.

24. The method of claim 11, wherein the surrounding tissue includes liver tissue.

25. The method of claim 11, wherein the object of interest is a tumor.
